(19) United States
(12) Patent Application Publication
(10) Pub. No.: US 2008/0139453 A1
(43) Pub. Date: Jun. 12, 2008

(54) Multiple emulsion compositions

(75) Inventors: Naohisa Yoshimi, Nishinomiya (JP); Hidekazu Tanaka, Toyonaka (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 12/001,526

(22) Filed: Dec. 12, 2007

Related U.S. Application Data
(60) Provisional application No. 60/874,533, filed on Dec. 12, 2006.

(51) Int. Cl. A61K 31/7008; A61K 8/58; A61P 17/00; A61K 47/30; A61K 38/02

(52) U.S. Cl. 514/2; 523/105; 514/62; 514/772.3

(57) ABSTRACT

The present invention relates to an O/W/O emulsion comprising; a) an external oil phase which comprises an emulsifying crosslinked siloxane elastomer and a solvent for the emulsifying crosslinked siloxane elastomer; and b) an O/W type primary emulsion which comprises an internal oil phase dispersed in an aqueous phase dispersed in the external oil phase wherein the aqueous phase comprises from about 0.01% to about 10% of a hydrophobically modified polymeric emulsifier, wherein when shear stress is applied to the O/W/O emulsion during spreading on skin, at least a portion of the primary emulsion is released from the O/W/O emulsion. The present invention also relates to an O/W/O emulsion composition comprising: a) from about 0.1% to about 15% of an emulsifying crosslinked siloxane elastomer; b) from about 0.01% to about 10% of a hydrophobically modified polymeric emulsifier; c) from about 1% to about 40% of a solvent for the emulsifying crosslinked siloxane elastomer; and d) water, wherein when shear stress is applied to the composition during spreading on skin, phase separation is occurred.

### CROSS REFERENCE TO RELATED APPLICATIONS

This application claims the benefit of U.S. Provisional Application No. 60/874,533 filed on Dec. 12, 2006.

### FIELD OF THE INVENTION

The present invention relates to multiple emulsion type skin care compositions. Such compositions are useful for delivering skin care actives in products with consumer acceptable aesthetics, in particular, for moisturizing, protecting and/or treating the skin and/or keratinous fibers.

### BACKGROUND OF THE INVENTION

Multiple emulsions of the O/W/O type have gained in importance in various industries including cosmetics, food products, and medicines, etc. O/W/O emulsions where O/W type emulsion is further emulsified in a continuous oil phase contain two oil phases, an internal oil phase and an external oil phase, separated from each other by an aqueous phase, and can be useful to formulate two or more oil-soluble substances which may be incompatible with one another in the emulsions. Also, it is useful to deliver easily oxidized oil-soluble compounds by incorporating them in the internal oil phase which is to be protected from the oxygen of the air.

Despite their advantages illustrated in the above, O/W/O emulsions have not been widely used due to their low emulsification stability over time. Destabilization of O/W/O emulsions is mainly caused by migration of the internal oil phase toward the external oil phase and merging with the external oil phase which finally brings phase separation.

In order to overcome these problems, various attempts have been made. One of such attempts is compounding specific materials in the water phase and/or an oil phase: U.S. Pat. No. 5,985,177 provides an O/W/O emulsion wherein an external oil phase contains an organophilic clay mineral. Another attempt is to employ special emulsifiers: U.S. Pat. No. 6,346,256 provides an O/W/O emulsion employing crosslinked siloxane elastomer comprising a polyoxyethylenated and/or polyoxypropylenated chain.

While delivery of specific skin care actives or compounds that can help to condition the skin and/or alleviate the damage caused by many extrinsic and intrinsic factors is of course important, consumer acceptance of the sensory and aesthetic aspects of a particular skin care composition is also important.

There are many effective and economic skin-conditioning agents including emollients, they nevertheless suffer from certain disadvantages. Use of emollients is limited as the level increases, greasy skin feel also increase. For example, petrolatum has long been used medicinally as skin conditioning agents, but only limitedly used in cosmetics due to its extreme greasiness and tackiness.

Many consumers dislike heavy, oily or greasy feeling compositions and prefer compositions that can provide smooth spreadability and water-like, fresh skin feel, with silky after-feel.

Based on the foregoing, there is a continuing need to formulate skin care compositions that can provide improved delivery of skin conditioning actives while also providing sensory and aesthetic benefits, especially as related to non-greasy and fresh feeling.

None of the existing art provides all of the advantages and benefits of the present invention.

### SUMMARY OF THE INVENTION

The present invention relates to an O/W/O emulsion comprising a) an external oil phase which comprises an emulsifying crosslinked siloxane elastomer and a solvent for the emulsifying crosslinked siloxane elastomer; and b) an O/W type primary emulsion which comprises an internal oil phase dispersed in an aqueous phase dispersed in the external oil phase, wherein the aqueous phase comprises from about 0.01% to about 10% of a hydrophobically modified polymeric emulsifier; wherein when shear stress is applied to the O/W/O emulsion during spreading on skin, at least a portion of the primary emulsion is released from the O/W/O emulsion.

The present invention also relates to an O/W/O emulsion comprising; a) an external oil phase which comprises an emulsifying crosslinked siloxane elastomer and a solvent for the emulsifying crosslinked siloxane elastomer; and b) an O/W type primary emulsion which comprises an internal oil phase dispersed in an aqueous phase dispersed in the external oil phase, wherein the aqueous phase comprises from about 0.01% to about 10% of a hydrophobically modified polymeric emulsifier; wherein when higher than about 1,000 sec−1, preferably 3,000 sec−1 of a shear rate is applied to the O/W/O emulsion, at least a portion of the primary emulsion is released from the O/W/O emulsion.

The present invention also relates to an O/W/O emulsion composition comprising: a) from about 0.1% to about 15% of an emulsifying crosslinked siloxane elastomer; b) from about 0.01% to about 10% of a hydrophobically modified polymeric emulsifier; c) from about 1% to about 40% of a solvent for the emulsifying crosslinked siloxane elastomer; and d) water, wherein when shear stress is applied to the composition during spreading on skin, phase separation is occurred.

The present invention also relates to an O/W/O emulsion composition comprising: a) from about 0.1% to about 15% of an emulsifying crosslinked siloxane elastomer; b) from about 0.01% to about 10% of a hydrophobically modified polymeric emulsifier; c) from about 1% to about 40% of a solvent for the emulsifying crosslinked siloxane elastomer; and d) water, wherein when higher than about 1,000 sec−1, preferably 3,000 sec−1 of a shear rate is applied to the composition, phase separation is occurred.

The present invention also relates to a method of preparing an O/W/O emulsion comprising: preparing an O/W type primary emulsion which comprises from about 0.01% to about 10% of a hydrophobically modified polymeric emulsifier; and dispersing the O/W type primary emulsion in a continuous external oil phase which comprises an emulsifying crosslinked siloxane elastomer and a solvent for the emulsifying crosslinked siloxane elastomer, wherein when higher than about 1,000 sec−1, preferably 3,000 sec−1 of a shear rate is applied to the composition, at least a portion of the primary emulsion is released from the O/W/O emulsion.

The present invention also relates to methods of using such compositions to regulate the condition of skin, said method comprising applying to the skin of a human in need of treatment.

These and other features, aspects, and advantages of the present invention will become evident to those skilled in the art from a reading of the present disclosure.

### DETAILED DESCRIPTION

While the specification concludes with the claims particularly pointing and distinctly claiming the invention, it is believed that the present invention will be better understood from the following description.

All percentages and ratios used herein are by weight of the total composition and all measurements made are at 25° C., unless otherwise designated.

As used herein, the “skin care products” are those used to treat or care for, or somehow moisturize, improve, or clean the skin. Products contemplated by the phrase “skin care products” include, but are not limited to moisturizers, personal cleansing products, occlusive drug delivery patches, nail polish, powders, wipes, hair conditioners, skin treatment emulsions, shaving creams and the like.

The term “ambient conditions” as used herein refers to surrounding conditions under about one atmosphere of pressure, at about 50% relative humidity, and at about 25° C. unless otherwise specified.

The compositions of the present invention can include, consist essentially of, or consist of, the components of the present invention as well as other ingredients described herein.

The term “keratinous tissue” as used herein, refers to keratin-containing layers disposed as the outermost protective covering of mammals (e.g., humans, dogs, cats, etc.) which includes, but is not limited to, skin, lips, hair, toenails, fingernails, cuticles, hooves, etc.

The term “safe and effective amount” as used herein, refers to an amount of a compound or composition sufficient to significantly induce a positive benefit, preferably a positive keratinous tissue appearance or feel benefit, or positive hair appearance or feel benefit, including independently or in combinations the benefits disclosed herein, but low enough to avoid serious side effects, i.e., to provide a reasonable benefit to risk ratio, within the scope of sound judgment of the skilled artisan.

The term “regulating skin condition” as used herein, refers to improving skin appearance and/or feel, for example, by providing a benefit, such as a smoother appearance and/or feel. The benefit may be a chronic benefit and may include one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc.

The term “primary emulsion” as used herein, refers to an O/W emulsion.

All percentages, parts and ratios are based upon the total weight of the skin care compositions of the present invention, unless otherwise specified. All such weights as they pertain to listed ingredients are based on the active level and, therefore, do not include carriers or by-products that may be included in commercially available materials, unless otherwise specified.

All publications cited herein are hereby incorporated by reference in their entirety.

The compositions of the present invention are useful for regulating the condition of skin and especially for regulating keratinous tissue condition.

The compositions of the present invention provide additional benefits, including stability, absence of significant (consumer-unacceptable) skin irritation and good aesthetics.

In the compositions of the present invention, upon application of shear force or shear stress, phase separation is occurred. The phase separation may be visibly observed in some compositions while it may not be visibly observed in other compositions. Without being bound by theory, at least a portion of the primary emulsion of the O/W/O emulsion may be separated from the external oil phase, and the separated primary emulsion may coalesce to form droplets within and/or upon the external oil phase. The perceptible release of the primary emulsion may be characterized by phase separation after milling according to the Milling Method provided in the Test Methods.

The external oil phase may be substantially evenly distributed upon the skin. Examples of shear force or shear stress include applying to the skin or other keratinous tissue, for example by smearing, rubbing, dabbing, wiping, etc. with a finger, hand, implement and/or a delivery enhancement device. The separate primary emulsion, especially when it is visible, may provide immediately benefits, including but not limited to, an immediate indication that the product is hydrating the keratinous tissue and/or an enhanced pleasant feel upon application. The separate primary emulsion may for example be rubbed into the skin or may be allowed to evaporate.

The compositions of the present invention is in the form of an O/W/O emulsion and contain an emulsifying crosslinked siloxane elastomer; a hydrophobically modified polymeric emulsifier; a solvent for the emulsifying crosslinked siloxane elastomers; and water.

The compositions of the present invention optionally contain a non-emulsifying crosslinked siloxane elastomer, an additional emulsifier, or an additional oil. The compositions also preferably contain one or more skin care actives. The nature of the actives and other ingredients depending on their nature, can be introduced into the aqueous phase or into one of the oil phases of the present emulsion.

The compositions herein may also include a wide variety of other ingredients. The compositions of the present invention are described in detail hereinafter.

### Emulsifying Crosslinked Siloxane Elastomers

An essential component of the present invention is an emulsifying crosslinked siloxane elastomer. The emulsifying crosslinked siloxane elastomer is present in the compositions of the present invention at concentrations of from about 0.1% to about 15%, preferably from about 0.2% to about 5%, most preferably from about 0.2% to about 2% by weight. The indicated percentages are understood to refer to amount of dry elastomer, as opposed to the total amount of elastomers and solvent, used for example for storage or shipping. The term “emulsifying,” as used herein, means crosslinked organopolysiloxane elastomer having at least one polyoxyalkylene (e.g., polyoxyethylene or polyoxypropylene) or polyglycerin moiety.

Emulsifying crosslinked siloxane elastomers in the present invention include those described in U.S. Pat. Nos. 5,412,002; 5,837,793 and 5,811,487. Non-limiting examples of useful emulsifying crosslinked siloxane elastomers are polyoxyalkylene-modified elastomers formed from divinyl compounds, particularly siloxane polymers with at least two free vinyl groups, reacting with Si—H linkages on a polysiloxane backbone. Such emulsifying crosslinked siloxane elastomers are supplied by Shin-Etsu (KSG-210, KSG-240, KSG-310, KSG-320 and KSG-330). Preferably, the elastomers are dimethyl polysiloxanes crosslinked by Si—H sites on a molecularly spherical MQ resin. Another preferred emulsifying crosslinked siloxane elastomers are siloxane polymers crosslinked with diallyl polyglycerin such as KSG-710 and KSG-810 available from Shin-Etsu.

### Solvent for an Emulsifying Crosslinked Siloxane Elastomer

The compositions of the present invention comprise a solvent for an emulsifying crosslinked siloxane elastomer. Concentrations of the solvent in the cosmetic compositions of the present invention will vary primarily with the type and amount of solvent and the emulsifying crosslinked siloxane elastomer employed. Concentrations of the solvent may be from about 1% to about 40%, preferably from about 4% to about 40%, more preferably from about 5% to about 30%, by weight of the composition.

The solvent, when combined with the emulsifying crosslinked siloxane elastomer particles, serves to suspend and swell the elastomer particles to provide an elastic, gel-like network or matrix. The solvent for the emulsifying cross-linked siloxane elastomer is liquid under ambient conditions, and in one embodiment has a low viscosity to provide for improved spreading on the skin.

The solvent for the emulsifying crosslinked siloxane elastomer may comprise one or more liquid carriers suitable for topical application to human skin. These liquid carriers may be organic, silicone-containing or fluorine-containing, volatile or non-volatile, polar or non-polar, provided that the liquid carrier forms a solution or other homogenous liquid or liquid dispersion with the selected emulsifying crosslinked siloxane elastomer at the selected siloxane elastomer concentration at a temperature of from about 28° C. to about 250° C., preferably from about 28° C. to about 100° C., preferably from about 28° C. to about 78° C. The solvent for the emulsifying crosslinked siloxane elastomer preferably has a solubility parameter of from about 3 to about 13 (cal/cm³)^{0.5}, more preferably from about 5 to about 11 (cal/cm³)^{0.5}, most preferably from about 5 to about 9 (cal/cm³)^{0.5}. Solubility parameters for the liquid carriers or other materials, and means for determining such parameters, are well known in the chemical arts. A description of solubility parameters and means for determining them are described by C. D. Vaughan, “Solubility Effects in Product, Package, Penetration and Preservation” 103 Cosmetics and Toiletries 47-69, October 1988; and C. D. Vaughan, “Using Solubility Parameters in Cosmetics Formulation”, 36 J. Soc. Cosmetic Chemists 319-333, September/October, 1988.

The solvent preferably includes volatile, non-polar oils; non-volatile, relatively polar oils; non-volatile, non-polar oils; and non-volatile paraffinic hydrocarbon oils; each discussed more fully hereinafter. The term “non-volatile” as used herein refers to materials that exhibit a vapor pressure of no more than about 0.2 mm Hg at 25° C. at one atmosphere and/or to materials that have a boiling point at one atmosphere of at least about 300° C. The term “volatile” as used herein refers to all materials that are not “non-volatile” as previously defined herein. The phrase “relatively polar” as used herein means more polar than another material in terms of solubility parameter; i.e., the higher the solubility parameter the more polar the liquid. The term “non-polar” typically means that the material has a solubility parameter below about 6.5 (cal/cm³)^{0.5}.

Non-limiting examples of suitable non-polar, volatile oil are disclosed in U.S. Pat. No. 4,781,917 issued to Luebbe et al. and include polydecanes such as isododecane and isodecane (e.g., Permethyl-99A, available from Presperse™ Inc.) and C7-C15 isoparaffins (e.g. the Isopar Series, from Exxon™ Chemicals); cyclomethicones of varying viscosities, e.g., Dow Corning™ 200, Dow Corning™ 244, Dow Corning™ 245, Dow Corning™ 344, and Dow Corning™ 345, Silicone Fluids, commercially available from G.E. Silicones, (e.g. SF-1204, SF-1202, GE 7207 and GE 7158); and SWS-03314 (commercially available from SWS Silicones™ Corp.).

Polar, non-volatile oils useful in the present invention include, but are not limited to, silicone oils; hydrocarbon oils; fatty alcohols; fatty acids; esters of mono and dibasic carboxylic acids with mono and polyhydric alcohols; polyoxyethylenes, polyoxypropylenes, mixtures of polyoxyethylene and polyoxypropylene ethers of fatty alcohols; and mixtures thereof. In one embodiment, the polar, non-volatile oil is selected from the group consisting of propoxylated ethers of C14-C18 fatty alcohols having a degree of propoxylation below about 50, esters of C2-C8 alcohols and C12-C26 carboxylic acids (e.g. ethyl myristate, isopropyl palmitate), esters of C12-C26 alcohols and benzoic acid (e.g. Finsolv™ TN supplied by Finetex™), diesters of C2-C8 alcohols and adipic, sebacic, and phthalic acids (e.g., diisopropyl sebacate, diisopropyl adipate, di-n-butyl phthalate), polyhydric alcohol esters of C6-C26 carboxylic acids (e.g., propylene glycol dicaprate/dicaprylate, propylene glycol isostearate); and mixtures thereof.

Examples of suitable non-volatile, non-polar oils include, but are not limited to, non-volatile polysiloxanes, paraffinic hydrocarbon oils, and mixtures thereof. The polysiloxanes useful in the present invention selected from the group consisting of polyalkylsiloxanes, polyarylsiloxanes, polyalkylarylsiloxanes, poly-ethersiloxane copolymers, and mixtures thereof. Examples of useful oils include Viscasil™ series (General Electric); Dow Corning 200 series (Dow Corning Corp.); SF 1075 methyl-phenyl fluid (General Electric) and 556 Cosmetic Grade Fluid (Dow Corning Corp.).

Non-volatile paraffinic hydrocarbon oils useful in the present invention are described in U.S. Pat. No. 5,019,375 issued to Tanner et al. and in 2003/0049212A1, and include mineral oils and branched-chain hydrocarbons such as Permethyl™ 102A, 103A and 104A (Permethyl Corporation); and Ethylflo™ 364 (Ethyl Corp.).

Additional solvents useful herein are described in U.S. Pat. No. 5,750,096 to Gerald J. Guskey et al., issued May 12, 1998.

### Hydrophobically Modified Polymeric Emulsifiers

The compositions of the present invention comprise a hydrophobically modified polymeric emulsifier. The hydrophobically modified polymeric emulsifier is present in the compositions of the present invention at concentrations of from about 0.01% to about 10%, preferably from about 0.1% to about 5%, most preferably from about 0.1% to about 2% by weight. Without being bound by theory, hydrophobically modified polymeric emulsifiers may operate by providing steric stabilization around oil droplets. When oil droplets stabilized with hydrophobically modified polymeric emulsifiers come near each other to a separation distance such that the adsorbed layers begin to overlap, they are repelled each other.

Hydrophobically modified polymeric emulsifiers having following non-limiting characteristics are desirable: (1) high solubility in aqueous phase of the polymeric moiety; (2) strong adsorption or anchoring of hydrophobic moiety to oil droplets; and (3) hydrodynamic thickness of the polymeric moiety enough to prevent close approach of oil droplets.

The polymer backbone of the hydrophobically modified polymeric emulsifier is hydrophilic and remains strongly hydrated in an aqueous phase. Any water soluble polymer may used as the backbone to form the hydrophobically modified polymeric emulsifier in the present invention. The polymer backbone is preferably nonionic, and may have a nonionic substituent such as methyl, hydroxyethyl, or hydroxypropyl so long as the polymer backbone is water soluble and can be hydrated in an aqueous phase.

Preferred polymer backbones are polysaccharides. Preferred polysaccharides include, but not limited to, a fructan-type saccharide having a fructosyl unit and a terminal glucosyl unit, and a starch-type saccharide having a glucosyl unit. Further preferred fructan-type saccharides include inulins, oligofructoses, fructo-oligosaccharides, partially hydrolysed inulins, levans and partially hydrolysed levans. The polysaccharides may have an average degree of polymerization ranging from 3-100. Another preferred starch-type saccharides include modified starches and starch hydrolysates.

The hydrophobic moiety of the hydrophobically modified polymeric emulsifier is substantially insoluble in the aqueous phase and has strong affinity to the oil droplet surface. The moiety can be provided by introduction of linear or branched C4-C22 alkyl, aryl alkyl, alkyl aryl groups or mixtures thereof on the polymer backbone. To ensure strong anchoring on the oil droplets, multiplicity of these hydrophobic moieties is preferable. The average number of hydrophobic moieties in the hydrophobically modified polymeric emulsifier varies depending on the number of a unit of the polymer backbone. The average number of hydrophobic moieties in the unit may be in the range of 0.01-2.0.

The hydrophobically modified polymeric emulsifier has hydrodynamic thickness of the polymeric moiety preferably in the range of 1-20 nm, more preferably, in the range of 5-10 nm.

The hydrophobically modified polymeric emulsifier can be prepared for example by conventional esterification reactions. For example, the hydrophobically modified polymeric emulsifier can be prepared by reaction of the appropriate polymer with an anhydride form of hydrophobic moiety in an appropriate solvent.

Preferred hydrophobically modified polymeric emulsifiers in the present invention are hydrophobically modified polysaccharide emulsifiers. Examples of such emulsifier include, but are not limited to, those described in U.S. Pat. No. 6,534,647 and US Publication No. 2004/0248761 A1. Suitable hydrophobically modified polymeric emulsifiers include Orafti Non-Food, Tienen's INUTEC series.

### Aqueous Phase

The compositions of the present invention comprise an aqueous phase comprising from about 20% to about 95%, preferably from about 50% to about 93%, more preferably from about 65% to about 90% by weight of the composition.

The aqueous phase may comprise water and/or other hydrophilic substances which exhibit limited solubility in an oil phase, including but not limited to water-soluble solvent, water-soluble sunscreens and other water-soluble skin care actives.

### Optional Ingredients

### Non-Emulsifying Crosslinked Siloxane Elastomers

The compositions of the present invention can optionally contain non-emulsifying crosslinked siloxane elastomers. The term “non-emulsifying,” as used herein, defines crosslinked organopolysiloxane elastomer from which polyoxyalkylene units or polyglycerin units are absent.

Non-limiting examples of non-emulsifying crosslinked siloxane elastomers used herein include dimethicone/vinyl dimethicone crosspolymers, supplied by a variety of suppliers including Dow Corning™ (DC 9040 and DC 9041), General Electric™ (SFE 839), Shin-Etsu™ (KSG-15, 16, 18 [dimethicone/phenyl vinyl dimethicone crosspolymer]), and Grant Industries (GRANSIL™ line of elastomers). Cross-linked siloxane elastomers useful in the present invention and processes for making them are further described in U.S. Pat. No. 4,970,252 to Sakuta, et al.; U.S. Pat. No. 5,760,116 to Kilgour, et al.; and U.S. Pat. No. 5,654,362 to Schulz, Jr., et al. issued Aug. 5, 1997. Additional crosslinked organopolysiloxane elastomers useful in the present invention are disclosed in Japanese Patent Application JP 61-18708, assigned to Pola Kasei Kogyo KK. In addition, suitable organopolysiloxane elastomer powders include vinyl dimethicone/methicone silesquioxane crosspolymers such as KSP-100, KSP-101, KSP-102, KSP-103, KSP-104, KSP-105 (Shin-Etsu™); hybrid silicone powders comprising a fluoroalkyl group, such as KSP-200 (Shin-Etsu™); and hybrid silicone powders comprising a phenyl group, such as KSP-300 (Shin-Etsu™) and DC-9506 (Dow Corning™).

In some embodiments, the composition may contain from about 0 to about 15%, preferably from about 0.1 to about 5%, most preferably from about 0.1 to about 2% of a non-emulsifying crosslinked siloxane elastomer by weight of the composition.

### Additional Emulsifiers

The compositions of the present invention can optionally contain an additional emulsifier. In some embodiments, the composition may contain from about 0% to about 5%, preferably from 0.01% to about 3% additional emulsifier, more preferably from about 0.1% to about 3% of an additional emulsifier by weight of the composition. The additional emulsifier if present helps disperse and suspend the internal oil phase within the aqueous phase. The additional emulsifier also can be used to help disperse and suspend the primary emulsion within the continuous external oil phase.

Known or conventional emulsifying agents can be used in the composition, provided that the selected emulsifying agent is chemically and physically compatible with components of the compositions of the present invention, and provides the desired dispersion characteristics. Suitable emulsifiers are disclosed in, for example, U.S. Pat. No. 3,755,560, issued Aug. 28, 1973, Dickert et al.; U.S. Pat. No. 4,421,769, issued Dec. 20, 1983, Dixon et al.; and McCutcheon's Detergents and Emulsifiers, North American Edition, pages 317-324 (1986). Non-limiting examples of nonionic emulsifiers are alkoxylated compounds based on C10-C22 fatty alcohols and acids, and sorbitan. These materials are available, for instance, from the Shell Chemical Company under the Neodol trademark, Copolymers of polyoxypropylene-polyoxyethylene, sold by the BASF Corporation under the Pluronic trademark, are sometimes also useful. Alkyl polyglycosides available from the Henkel Corporation may also be utilized for purposes of this invention. Anionic type emulsifiers or surfactants include fatty acid soaps, sodium lauryl sulphate, sodium lauryl ether sulphate, alkyl benzene sulphonate, mono- and di-alkyl acid phosphates and sodium fatty acyl isethionate. Amphoteric emulsifiers or surfactants include such materials as dialkylamine oxide and various types of betaines (such as cocamidopiopyl betaine).

In one embodiment, the additional emulsifier is a silicone emulsifier. A wide variety of silicone emulsifiers is useful herein. These silicone emulsifiers are typically organically modified siloxanes, also known to those skilled in the art as silicone surfactants. Useful silicone emulsifiers include dimethicone copolyols. These materials are polydimethyl siloxanes which have been modified to include polyether side chains such as polyethylene oxide chains, polypropylene oxide chains, polyglycerine chains, mixtures of these chains, and polyether chains containing moieties derived from both ethylene oxide and propylene oxide. Other examples include alkyl-modified dimethicone copolyols, i.e., compounds which contain C2-C30 pendant side chains. Still other useful dimethicone copolyols include materials having various cationic, anionic, amphoteric, and zwitterionic pendant moieties.

### Other Oils

The compositions of the present invention may contain at least one other oil which is not a solvent for the emulsifying siloxane elastomer. In the present invention, wide range of oils from polar oils to non polar oils can be used as other oil for the internal and/or external oil phase component. Other oils in the present invention include, but are not limited to, hydrocarbon oils and waxes, fatty alcohol and fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, salts, isomers and derivatives thereof, and combinations thereof.

Non-limiting examples of hydrocarbon oils and waxes suitable for use herein include, but are not limited to, petrolatum, mineral oil, micro-crystalline waxes, polyalkenes, paraffins, isoparaffin, branched-chain light paraffin, polyethylene, squalane, perhydrosqualene, and ester oils such as isopropyl myristate, cetyl isoocatanoate, and glyceryl trioctanoate, and combinations thereof.

### Skin Care Actives

The compositions of the present invention may include at least one skin care active. Without being bound by theory, it is believed the present compositions provide versatility in formulating a variety of actives.

In any embodiment of the present invention, however, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed.

Vitamin B₃ Compounds

Vitamin B₃ compounds such as niacinamide are a preferred skin care active for use herein. The present invention preferably includes from about 0.1% to about 30%, more preferably from about 1% to about 20%, even more preferably from about 2% to about 10% of a vitamin B₃ compound.

As used herein, “vitamin B₃ compound” means a compound having the formula:

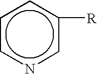

wherein R is —CONH₂ (i.e., niacinamide), —COOH (i.e., nicotinic acid) or —CH₂OH (i.e., nicotinyl alcohol); derivatives thereof; and salts of any of the foregoing. Exemplary derivatives of the foregoing vitamin B₃ compounds include nicotinic acid esters, including non-vasodilating esters of nicotinic acid (e.g., tocopheryl nicotinate), nicotinyl amino acids, nicotinyl alcohol esters of carboxylic acids, nicotinic acid N-oxide and niacinamide N-oxide.

Whitening Agents

The present compositions may contain a whitening agent. The whitening agent useful herein refers to active ingredients that not only alter the appearance of the skin, but further improve hyperpigmentation as compared to pre-treatment. Useful whitening agents useful herein include ascorbic acid compounds, vitamin B₃ compounds, azelaic acid, butyl hydroxy anisole, gallic acid and its derivatives, hydroquinone, kojic acid, arbutin, mulberry extract, undecylenoyl phenylalanine, cetyl pyridinium chloride, glycyrrhizic acid, tetrahydrocurcumin, and mixtures thereof. Use of combinations of whitening agents is also believed to be advantageous in that they may provide whitening benefit through different mechanisms.

When used, the compositions preferably contain from about 0.1% to about 10%, more preferably from about 0.2% to about 5%, by weight of the composition, of a whitening agent.

Ascorbic acid compounds are useful whitening agents, and have the formula (I):

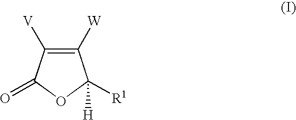

wherein V and W are independently —OH; R¹ is —CH(OH)—CH₂OH; and salts thereof. Preferably, the ascorbic acid compound useful herein is an ascorbic acid salt or derivative thereof, such as the non-toxic alkali metal, alkaline earth metal and ammonium salts commonly known by those skilled in the art including, but not limited to, the sodium, potassium, lithium, calcium, magnesium, barium, ammonium and protamine salts which are prepared by methods well known in the art.

Undecylenoyl Phenylalanine is the substituted amino acid that is also suitable for use herein as a whitening agent. It is available under the trade name Sepiwhite, from Seppic.

Peptides

Peptides, including but not limited to, di-, tri-, tetra-, and pentapeptides and derivatives thereof, may be included in the compositions of the present invention in amounts that are safe and effective. As used herein, “peptides” refers to both the naturally occurring peptides and synthesized peptides. Also useful herein are naturally occurring and commercially available compositions that contain peptides.

When included in the present compositions, peptides are preferably included in amounts of from about 1×10⁻⁶% to about 10%, more preferably from about 1×10⁻⁶% to about 0.1%, even more preferably from about 1×10⁻⁵% to about 0.01%, by weight of the composition.

Sugar Amines

The compositions of the present invention may include a safe and effective amount of a sugar amine, which are also known as amino sugars. As used herein, “sugar amine” refers to an amine derivative of a six-carbon sugar. Examples of sugar amines that are useful herein include glucosamine, N-acetyl glucosamine, mannosamine, N-acetyl mannosamine, galactosamine, N-acetyl galactosamine. Preferred for use herein is glucosamine. Additionally, combinations of two or more sugar amines may be used.

When included in the present compositions, a sugar amine is preferably included in amounts of from about 0.001% to about 20%, more preferably from about 1% to about 10%, even more preferably from about 2% to about 5%, by weight of the composition.

### Skin Conditioning Agent

Optionally, the composition of the present invention can further comprise a skin conditioning agent. These agents may be selected from humectants, exfoliants or emollients. The amount of skin-condition agent may range from about 1% to about 60%, preferably from about 2% to about 50%, more preferably from about 5% to about 40%, by weight of the composition.

Humectants are polyhydric alcohols intended for moisturizing, reducing scaling and stimulating removal of built-up scale from the skin. Typical polyhydric alcohols include polyalkylene glycols and more preferably alkylene polyols and their derivatives. Illustrative are propylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin and mixtures thereof. Most preferably the humectant is glycerin.

Exfoliants according to the present invention may be selected from C2-C30 alpha-hydroxycarboxylic acids, beta-hydroxycarboxylic acids and salts of these acids. Most preferred are glycolic, lactic and salicylic acids and their ammonium salts.

When the conditioning agent is an emollient it may be selected from hydrocarbons, fatty acids, fatty alcohols and esters.

### Oil-Soluble Compounds

The term “oil-soluble”, as used herein, means when calculating inorganic/organic balance, organic portion is equal or bigger than inorganic portion. The oil-soluble compounds may be selected from oil-soluble vitamin compounds, oil-soluble terpene alcohols, phytosterol and derivatives thereof.

The amount of an oil-soluble compound may range from about 0.01% to about 10%, preferably, about 0.05% to about 5%, more preferably from about 0.1% to about 3%, by weight of the composition.

A number of vitamins known by those in the art for providing various skin benefits are oil-soluble and some or all of their derivatives are oil-soluble. Non-limiting examples of such oil-soluble vitamin compounds include retinoids, vitamin C (e.g. ascorbyl palmitate), vitamin D, vitamin K, vitamin E, and mixtures thereof. Preferred for use herein are retinoids, vitamin E, and mixtures thereof.

Oil-soluble terpene alcohols that are useful herein include farnesol, derivatives of farnesol, isomers of farnesol, geraniol, derivatives of geraniol, isomers of geraniol, phytantriol, derivatives of phytantriol, isomers of phytantriol, and mixtures thereof. Preferred for use herein is farnesol.

Phytosterol and derivatives thereof are known for providing skin lightening benefits. Non-limiting examples of oil-soluble phytosterol derivatives include β-sitosterol, campesterol, brassicasterol, lupenol, α-spinasterol, stigmasterol, their derivatives, and combinations thereof.

### Sunscreen Agents

The compositions of the subject invention may optionally contain a sunscreen agent. Suitable sunscreen agents may be organic or inorganic.

Inorganic sunscreen agents useful herein include the following metallic oxides; titanium dioxide, zinc oxide, zirconium oxide, iron oxide, and mixtures thereof.

Organic sunscreen agents useful herein include homosalate, octocrylene, 2-ethylhexyl-p-methoxycinnamate, phenyl benzimidazole sulfonic acid, 2-hydroxy-4-methoxybenzophenone (Benzophenone-3), 2-ethylhexyl-salicylate, and mixtures thereof.

When included in the present compositions, the sunscreens are preferably included in amounts of from about 0.1% to about 20%, preferably from about 0.5% to about 10%, more preferably from about 1% to about 5%, by weight of the composition. Exact amounts will vary depending upon the sunscreen or sunscreens chosen and the desired Sun Protection Factor (SPF).

### Thickening Agents

The compositions of the present invention, in some embodiments, may further include one or more thickening agents.

Nonlimiting classes of thickening agents include those selected from the following: carboxylic acid polymers, crosslinked polyacrylate polymers, polyacrylamide polymers, polysaccharides and gums.

When present, the composition preferably includes from about 0.01% to about 5%, more preferably from about 0.1% to about 4%, and still more preferably from about 0.1% to about 3%, by weight of the composition of the thickening agent.

### Other Optional Ingredients

A variety of additional ingredients can be incorporated into the compositions of the present invention. Nonlimiting examples of these additional ingredients includes; particular materials to modify skin feel or appearance; anti-acne actives; oil-soluble beta-hydroxy acids such as salicylic acid and derivatives thereof; chelators; flavonoid compounds; anti-inflammatory agents; anti-cellulite agents; desquamation actives; anti-oxidant/radical scavengers; tanning actives; skin soothing or skin healing actives such as panthenoic acid derivatives (including panthenol, dexpanthenol, ethyl panthenol), aloe vera, allantoin, bisabolol, and dipotassium glycyrrhizinate; antimicrobial or antifungal actives.

### Composition Preparation

The O/W/O emulsions of the present invention may be prepared by conventional preparation of O/W/O emulsions. Methods of preparing O/W type emulsions and/or O/W/O type emulsions are not restricted in particular as long as a stable emulsion can be obtained thereby.

In one embodiment, a method of preparing the O/W/O type emulsion comprises:

preparing an external oil phase preparation by mixing an emulsifying crosslinked siloxane elastomer, a solvent for the emulsifying crosslinked siloxane elastomer and an optional ingredient for the external oil phase;

separately preparing an O/W type emulsion by dispersing an internal oil phase which comprises oil and an optional ingredient for the internal oil phase within an aqueous phase which comprises water, a hydrophobically modified polymeric emulsifier and an optional ingredient for the aqueous phase, and

dispersing the O/W type emulsion within the external oil phase.

In another embodiment, a method of preparing the O/W/O type emulsion comprises:

preparing an external oil phase preparation by mixing an emulsifying crosslinked siloxane elastomer, a solvent for the emulsifying crosslinked siloxane elastomer, and optional ingredients for the external oil phase;

separately preparing an O/W type emulsion by preparing a concentrated O/W type emulsion by mixing oil, a hydrophobically modified polymeric emulsifier, a water-soluble solvent such as glycerin and/or water, and an optional ingredient for an internal oil phase, and by mixing the concentrated O/W type emulsion, water and optional ingredients for an aqueous phase; and

dispersing the O/W type emulsion within the external oil phase.

### Products for Topical Use

In one preferred embodiment, the O/W/O emulsion of the present invention is an O/W/O emulsion comprising an external oil phase which comprises an emulsifying crosslinked siloxane elastomer and a solvent for the emulsifying crosslinked siloxane elastomer; and an O/W type primary emulsion which comprises an internal oil phase dispersed in an aqueous phase comprising from about 0.01% to about 10% of a hydrophobically modified polymeric emulsifier dispersed in the external oil phase. In this embodiment, when shear stress is applied to the composition during spreading on skin, the primary emulsion is released from the O/W/O emulsion.

In this embodiment, the emulsion may comprise from about 20% to about 95%, preferably from about 50% to about 93%, more preferably from about 70% to about 90% of an aqueous phase.

In this embodiment, the emulsion may contain at least one compound selected from a group consisting skin care actives, skin conditioning agents, oil-soluble compounds, sunscreen agents and mixtures thereof.

In this embodiment, the internal oil phase may contain a solvent for the emulsifying siloxane elastomer, and/or an oil-soluble compound such as petrolatum which is incompatible with a solvent for the emulsifying siloxane elastomer.

In another preferred embodiment, the emulsion composition of the present invention is an O/W/O type emulsion composition which comprises from about 0.1% to about 15% of an emulsifying crosslinked siloxane elastomer; from about 0.01% to about 10% of a hydrophobically modified polysaccharide emulsifier; from about 1% to about 40% of a solvent for the emulsifying crosslinked siloxane elastomer; and water. In this embodiment, when shear stress is applied to the composition during spreading on skin, phase separation is occurred.

In this embodiment, the phase separation may be visibly observed and can provide a water-splash sensation during spreading or rubbing upon the skin. For example, at the initial application to the skin but before spreading upon the skin, the composition may be an intact multiple emulsion in the form of a lotion, gel or cream. During skin application, the multiple emulsion is expected to get from about 10,000 sec⁻¹ to about 100,000 sec⁻¹ of shear rate upon spreading. Such shear rate is believed to break the binding of the aqueous phase to the external oil phase, thereby releasing the primary emulsion from the multiple emulsion. This provides good consumer sensory benefit, as the aqueous phase so released is perceptible to the touch as well as visually.

In this embodiment, the composition may further comprise from about 0.1% to about 15% of a non-emulsifying crosslinked siloxane elastomer.

In this embodiment, the composition may further comprises at least one compound selected from a group consisting skin care actives, skin conditioning agents, oil-soluble compounds, sunscreen agents and mixtures thereof.

In another preferred embodiment, the emulsion of the present invention further comprises an additional emulsifier and/or a thickening agent. In this embodiment, the phase separation may not be visibly observed depending on the level of the additional emulsifier and/or the thickening agent.

In this embodiment, the emulsion preferably contains a silicone emulsifier.

Without being bound by theory, the amount of primary emulsion released from the O/W/O emulsion and the rate at which the primary emulsion is released from the O/W/O emulsion can be controlled, depending upon how the external oil phase is bonded to the aqueous phase in the O/W/O emulsion. In addition, it is also believed that the amount of the primary emulsion released from the O/W/O emulsion and the rate at which it is released from the O/W/O emulsion can be controlled, for example, by incorporating an additional emulsifier in the aqueous phase and/or the external oil phase, by changing the level of the emulsifying crosslinked siloxane elastomer within the claimed range, and by varying the aqueous phase/external oil phase ratio.

### Method of Use

Applicants have found that the compositions of the present invention are useful in a variety of applications directed to enhancement of mammalian skin. The methods of use for the compositions disclosed and claimed herein include, but are not limited to: 1) methods of increasing the substantivity of a cosmetic to skin; 2) methods of moisturizing skin; 3) methods of improving the natural appearance of skin; 4) methods of applying a color cosmetic to skin; 5) methods of preventing, retarding, and/or treating wrinkles; 6) methods of providing UV protection to skin; 7) methods of preventing, retarding, and/or controlling the appearance of oil; 8) methods of modifying the feel and texture of skin; 9) methods of providing even skin tone; 10) methods of preventing, retarding, and/or treating the appear of spider vessels and varicose veins; 11) methods of masking the appearance of vellus hair on skin; and 12) methods of concealing blemishes and/or imperfections in human skin, including acne, age spots, freckles, moles, scars, under eye circles, birth marks, post-inflammatory hyperpigmentation, etc. Each of the methods discussed herein involve topical application of the claimed compositions to skin.

### Test Methods

### Milling Method

The primary emulsion release is measured by a milling method. This method involves the bulk milling of the sample emulsion (“Sample”) to provide higher than about 1,000 sec⁻¹ of a shear rate evenly to a Sample, yield a phase separation, and measure weight or amount of the separated phase. The amount of sample and the milling time can be adjusted depending on an equipment type.

As an Example, the milling method involves the bulk milling of a 30 g Sample in 50 mL beaker using an Ultra Turrax T25 mixer with a S 25 KR-18G dispersing element available from IKA Works, Wilmington, N.C. The method is conducted at a temperature of approximately 25° C. The Sample is milled for about 1 minute at a speed of either about 13,500 rpm (which corresponds to a shear rate of about 30,000 s−1) or about 20,500 rpm (which corresponds to a shear rate of about 45,000 s−1). Optionally, a Sample may be milled at a speed of 8,000 rpm (which corresponds to a shear rate of about 17,500 s−1). During the 1 minute of milling, the beaker may be gently (i.e., reciprocating motion of no more than about 1 Hz) moved by hand in a direction parallel to the rotor axis of the mixer. After no more than 5 minutes after milling is ended, phase separation is observed. The primary emulsion is removed from the beaker using standard separation techniques. The separated primary emulsion is weighed.

### Stability Test

Stability of emulsions is tested by measuring viscosity, appearance, odor or microscopic visual check of emulsions after storage for three months or more at 40° C. or for one month or more at 50° C.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

O/W/O emulsion skin care products are prepared by conventional methods from the following components.

### Examples 1-7

| | | | | | | | |
|---|---|---|---|---|---|---|---|
| Ingredient (Wt %) | EX 1 | EX 2 | EX 3 | EX 4 | EX 5 | EX 6 | EX 7 |

Phase A: External Oil Phase

| | | | | | | | |
|---|---|---|---|---|---|---|---|
| KSG-15 *1 | — | — | — | — | 1.000 | — | — |
| DC9040 *2 | — | — | 3.000 | 3.000 | — | 3.000 | 3.000 |
| KSG-210 *3 | 5.000 | 5.000 | 2.750 | 2.500 | 2.500 | 2.750 | 2.750 |
| Dimethicone *4 | 5.000 | 5.000 | 4.000 | 4.000 | — | 4.000 | 4.000 |
| Cyclomethicone *5 | 5.000 | 5.000 | 3.000 | 3.000 | 9.000 | 3.000 | 3.000 |
| KF-6104 *6 | — | 0.200 | — | — | — | — | 0.500 |
| Polymethylsilsesquioxane*7 | — | — | 4.000 | — | — | 4.000 | 4.000 |

Phase B: Internal Oil Phase

| | | | | | | | |
|---|---|---|---|---|---|---|---|
| Petrolatum | 1.800 | — | 1.800 | — | — | 1.800 | 1.800 |
| Jojoba Oil | 2.200 | — | 2.200 | — | — | 2.200 | 2.200 |
| DC1503 *8 | — | — | — | 4.000 | 1.000 | — | — |
| Squalane | — | — | — | — | 2.000 | — | — |

Phase C: Aqueous phase

| | | | | | | | |
|---|---|---|---|---|---|---|---|
| Niacinamide USP | 4.000 | 4.000 | 3.600 | 4.000 | 4.000 | 4.000 | 4.000 |
| D-Pantenol | 0.500 | 0.500 | 0.450 | 0.500 | 0.500 | 0.500 | 0.500 |
| Glycerin | 10.000 | 10.000 | 10.000 | 10.000 | 10.000 | 10.000 | 10.000 |
| N-Acetyl Glucosamine | — | — | 2.000 | — | — | — | — |
| Pentylene Glycol | 2.000 | 2.000 | 2.000 | 2.000 | 2.000 | 2.000 | 2.000 |
| Methylparaben | 0.200 | 0.200 | 0.200 | 0.200 | 0.200 | 0.200 | 0.200 |
| Eethylparaben | 0.050 | 0.050 | 0.050 | 0.050 | 0.050 | 0.050 | 0.050 |
| Sodium Benzoate | 0.070 | 0.070 | 0.070 | 0.070 | 0.070 | 0.070 | 0.070 |
| EDTA-2NA | 0.100 | 0.100 | 0.100 | 0.100 | 0.100 | 0.100 | 0.100 |
| Sodium Citrate | 0.200 | 0.200 | — | — | — | 0.200 | 0.200 |
| Citric acid, Anhydrous | 0.030 | 0.030 | — | — | — | 0.015 | 0.015 |
| Sodium Chloride | 0.500 | 0.500 | — | — | — | 0.500 | 0.500 |
| Keltrol *9 | — | — | 0.020 | 0.020 | 0.020 | — | — |
| Cosmedia SP (Na | — | — | 0.200 | — | — | — | — |
| Polyacrylate)*10 | | | | | | | |
| Carbopol 831 or 981 *11 | — | — | — | 0.075 | — | — | — |
| Ultrez 21 *12 | — | — | — | — | 0.150 | — | — |
| Triethanolamine | — | — | — | 0.086 | 0.120 | — | — |
| Inutec SP1 *13 | 0.400 | 0.400 | 0.400 | 0.267 | 0.234 | 0.200 | 0.200 |
| Purified Water | q.s. to | q.s. to | q.s. to | q.s. to | q.s. to | q.s. to | q.s. to |
| | 100 | 100 | 100 | 100 | 100 | 100 | 100 |

| | | | | | |
|---|---|---|---|---|---|
| Ingredient (Wt %) | EX 8 | EX 9 | EX 10 | EX 11 | EX 12 |

Phase A: External Oil Phase

| | | | | | |
|---|---|---|---|---|---|
| KSG-15 *1 | 1.500 | 1.500 | 1.500 | 1.500 | 1.500 |
| KSG-210 *3 | — | — | — | — | 3.000 |
| KSG-240 *3-1 | 3.000 | 3.000 | 3.000 | 3.000 | — |
| Dimethicone *4 | — | — | — | — | 4.000 |
| Cyclomethicone *5 | 9.000 | 9.000 | 9.000 | 9.000 | 4.000 |
| KF-6104 *6 | 0.500 | — | 0.500 | 0.750 | — |
| KF-6105 *6-1 | — | 0.500 | — | — | — |
| Titanium dioxide | — | — | — | 0.200 | — |
| Cerapure H3C *7-1 | 0.750 | 0.750 | 0.750 | 0.750 | — |

Phase B: Internal Oil Phase

| | | | | | |
|---|---|---|---|---|---|
| DC1503 *8 | 1.500 | — | 1.500 | 1.500 | — |
| Squalane | 0.500 | — | 0.500 | 0.500 | — |
| Indole Acetic Acid | — | 0.200 | — | — | — |
| Eldew SL-205 *8-1 | — | 2.000 | 2.000 | 0.350 | 1.900 |
| BHT | — | 0.500 | 0.500 | — | — |
| Tocopheryl Acetate | — | 2.000 | 2.000 | — | 1.100 |
| Rhodysterol *8-2 | — | — | — | 0.500 | — |

Phase C: Aqueous phase

| | | | | | |
|---|---|---|---|---|---|
| Niacinamide USP | — | — | — | — | 4.000 |
| D-Pantenol | 0.500 | 0.500 | 0.500 | 0.500 | 0.500 |
| Glycerin | 20.000 | 20.000 | 20.000 | 20.000 | 10.000 |
| Pentylene Glycol | 2.000 | 2.000 | 2.000 | 2.000 | 2.000 |
| Methylparaben | 0.200 | 0.200 | 0.200 | 0.200 | 0.200 |
| Eethylparaben | 0.050 | 0.050 | 0.050 | 0.050 | 0.050 |
| Sodium Benzoate | 0.070 | 0.070 | 0.070 | 0.070 | 0.070 |
| EDTA-2NA | 0.100 | 0.100 | 0.100 | 0.100 | 0.100 |
| Carbopol 831 or 981 *11 | 0.200 | 0.200 | 0.200 | 0.200 | 0.200 |
| Ultrez 21 *12 | 0.150 | 0.150 | 0.150 | 0.150 | 0.150 |
| Triethanolamine | 0.350 | 0.350 | 0.350 | 0.350 | 0.350 |
| Inutec SP1 *13 | 0.020 | 0.141 | 0.020 | 0.200 | 0.200 |
| Purified Water | q.s. to | q.s. to | q.s. to | q.s. to | q.s. to |
| | 100 | 100 | 100 | 100 | 100 |

*1. 5% Dimethicone/Vinyl Dimethicone Crosspolymer in Dimethicone. Available from Shin-Etsu
*2. 12.5% Dimethicone Crosspolymer in Cyclopentasiloxane. Available from Dow Corning
*3. 25% Dimethicone PEG-10/15 Crosspolymer in Dimethicone. Available from Shin-Etsu
*3-1 KSG240-1: 5% Dimethicone/Vinyl Dimethicone Crosspolymer in Cyclomethicone. Available from Shin-Etsu
*4. KF96A (6cs). Available from Shin-Etsu
*5. E.g., SH245, DC245 or SF1202.
*6. Polyglyceryl-3 Polydimethylsiloxyethyl Dimethicone. Available from Shin-Etsu.
*6-1 Lauryl Polyglyceryl-3 Polydimethylsiloxyethyl Dimethicone. Available from Shin-Etsu.
*7. E.g., Tospearl 145A or CF 600. Available from GE Toshiba Silicone
*7-1 Polyethylene. Available from Shamrock
*8. Dimethicone/Dimethiconol. Aavailable from Dow Corning
*8-1 Isopropyl Lauroyl Sarcosinate. Available from Ajinomoto
*8-2 Mixture of Propylene Glycol Diethylhexanoate and Gelidium Cartilagineum Extract Available from BiotechMarine
*9. Xanthan Gum. Available from Kelco
*10. Carbomer. Available from Noveon
*11. Available from Cognis
*12. Acrylates/C10-30 Alkyl Acrylate Crosspolymer. Available from Noveon
*13. Inulin Lauryl Carbamate. Available from Orafti Non-Food

In separate suitable containers are added the ingredients of Phase A, Phase B and Phase C, and each phase is mixed using a suitable mixer (e.g., Anchor blade, propeller blade, IKA T25). When each phase is homogenous, slowly add Phase B to Phase C while mixing Phase C with a suitable mixer (e.g., Anchor blade, propeller blade, IKA T25). When batch is homogenous, slowly add the mixture of Phase B and Phase C to Phase A while mixing Phase A with a suitable mixer (e.g., Anchor blade, propeller blade, IKA T25). Maintain mixing until batch is uniform. Pour product into suitable containers.

Select examples tested according to the Milling Method by using an Ultra Turrax T25 mixer with a S 25 KR-18G dispersing element available from IKA Works provide the following primary emulsion release:

| | Shear rate | Comparative | | |
|---|---|---|---|---|
| | (sec−1) | Example* | Example 6 | Example 7 |
| 8,000 rpm | 17,582 | 0.0 g | 0.0 g | 0.0 g |
| 13,500 rpm | 29,670 | 0.0 g | 8.86 g | 0.0 g |
| 20,500 rpm | 45,054 | 0.0 g | 11.41 g | 1.28 g |

*The Comparative Example is the commercially available Regenerist Daily Regenerating Serum available from The Procter & Gamble Company.

It is understood that the foregoing detailed description of examples and embodiments of the present invention are given merely by way of illustration, and that numerous modifications and variations may become apparent to those skilled in the art without departing from the spirit and scope of the invention; and such apparent modifications and variations are to be included in the scope of the appended claims.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An O/W/O emulsion composition comprising:
a) from about 0.1% to about 15% of an emulsifying crosslinked siloxane elastomer;
b) from about 0.01% to about 10% of a hydrophobically modified polymeric emulsifier;
c) from about 1% to about 40% of a solvent for the emulsifying crosslinked siloxane elastomer; and
d) water,
wherein when shear stress is applied to the composition during spreading on skin, phase separation occurs.

2. The O/W/O emulsion composition according to claim 1, further comprising from about 0.1% to about 15% of a non-emulsifying crosslinked siloxane elastomer.

3. The O/W/O emulsion composition according to claim 1, wherein said hydrophobically modified polymeric emulsifier is a hydrophobically modified polysaccharide emulsifier.

4. The O/W/O emulsion composition according to claim 3, wherein said hydrophobically modified polysaccharide emulsifier is selected from the group consisting of a fructan-based emulsifier and a starch-based emulsifier.

5. The O/W/O emulsion composition according to claim 1, further comprising at least one compound selected from a group consisting skin care actives, skin conditioning agents, oil-soluble compounds, sunscreen agents and mixtures thereof.

6. The O/W/O emulsion composition according to claim 5, wherein said skin care actives are selected from the group consisting of vitamin B₃ compounds, whitening actives, peptides, sugar amines and mixtures thereof.

7. An O/W/O emulsion composition comprising:
a) from about 0.1% to about 15% of an emulsifying crosslinked siloxane elastomer;
b) from about 0.01% to about 10% of a hydrophobically modified polymeric emulsifier;
c) from about 1% to about 40% of a solvent for the emulsifying crosslinked siloxane elastomer; and
d) water,
wherein when higher than about 1,000 sec⁻¹ of a shear rate is applied to the composition, phase separation occurs.

8. An O/W/O emulsion comprising;
a) an external oil phase which comprises an emulsifying crosslinked siloxane elastomer and a solvent for the emulsifying crosslinked siloxane elastomer; and
b) an O/W type primary emulsion which comprises an internal oil phase dispersed in an aqueous phase dispersed in the external oil phase, wherein the aqueous phase comprises from about 0.01% to about 10% of a hydrophobically modified polymeric emulsifier,
wherein when shear stress is applied to the O/W/O emulsion during spreading on skin, at least a portion of the primary emulsion is released from the O/W/O emulsion.

9. The O/W/O emulsion according to claim 8, wherein said internal oil phase comprises a compound incompatible with the solvent for the emulsifying crosslinked siloxane elastomer.

10. The O/W/O emulsion according to claim 8, wherein said hydrophobically modified polymeric emulsifier is selected from the group consisting of a fructan-based emulsifier and a starch-based emulsifier.

11. The O/W/O emulsion according to claim 8, further comprising from about 0.1% to about 15% of a non-emulsifying crosslinked siloxane elastomer.

12. The O/W/O emulsion according to claim 8, further comprising at least one compound selected from a group consisting skin care actives, skin conditioning agents, oil-soluble compounds, sunscreen agents and mixtures thereof.

13. The O/W/O emulsion according to claim 12, wherein said skin care actives are selected from the group consisting of vitamin B₃ compounds, whitening actives, peptides, sugar amines and mixtures thereof.

14. The O/W/O emulsion according to claim 8, wherein said external oil phase comprises from about 0.1% to about 15% of an emulsifying crosslinked siloxane elastomer.

15. An O/W/O emulsion comprising;
a) an external oil phase which comprises an emulsifying crosslinked siloxane elastomer and a solvent for the emulsifying crosslinked siloxane elastomer; and
b) an O/W type primary emulsion which comprises an internal oil phase dispersed in an aqueous phase dispersed in the external oil phase, wherein the aqueous phase comprises from about 0.01% to about 10% of a hydrophobically modified polymeric emulsifier,
wherein when higher than about 10,000 shear rate/sec of shear stress is applied to the O/W/O emulsion, at least a portion of the primary emulsion is released from the O/W/O emulsion.

16. A method of preparing an O/W/O emulsion comprising:
a) preparing an O/W type primary emulsion which comprises from about 0.01% to about 10% of a hydrophobically modified polymeric emulsifier; and
b) dispersing the O/W type primary emulsion in a continuous external oil phase which comprises an emulsifying crosslinked siloxane elastomer and a solvent for the emulsifying crosslinked siloxane elastomer,
wherein when higher than about 1,000 sec⁻¹ of a shear rate is applied to the composition, at least a portion of the primary emulsion is released from the O/W/O emulsion.
